(19) United States
(12) Patent Application Publication
(10) Pub. No.: US 2021/0315521 A1
(43) Pub. Date: Oct. 14, 2021

(54) MODULAR CARDIAC PATIENT TREATMENT AND MONITORING

(71) Applicant: West Affum Holdings Corp., Grand Cayman (KY)

(72) Inventor: Brian D. Webster, Mercer Island, WA (US)

(21) Appl. No.: 17/192,496

(22) Filed: Mar. 4, 2021

Related U.S. Application Data
(60) Provisional application No. 63/008,612, filed on Apr. 10, 2020.

(51) Int. Cl. A61B 5/00; A61B 5/28; A61B 5/145; A61B 5/318; A61M 5/142; A61M 5/172; A61N 1/39

(52) U.S. Cl. CPC A61B 5/4836; A61B 5/6804; A61B 5/28; A61B 5/14532; A61N 1/39; A61B 5/0015; A61B 5/318; A61M 5/142; A61M 5/1723; A61B 5/0004

(57) ABSTRACT

Technologies and implementations for a wearable healthcare system having a reconfigurable medical device integrated with a support structure included in the wearable healthcare system. The technologies and implementations facilitate configuring and reconfiguring the medical device based, at least, on the health condition of a person.

### RELATED APPLICATION

This application claims benefit of priority to U.S. Provisional Patent Application Ser. No. 63/008,612, filed on Apr. 10, 2020, titled MODULAR CARDIAC PATIENT TREATMENT AND MONITORING, which is incorporated herein by reference in its entirety for all purposes.

### INFORMATION

Unless otherwise indicated herein, the approaches described in this section are not prior art to the claims in this application and are not admitted to be prior art by inclusion in this section.

Technology has contributed to improvements in healthcare. Some examples include healthcare related devices that may be mobile and personal. Mobile and personal healthcare devices may include Wearable Medical Devices (WMDs). Some WMDs may include medical devices that facilitate monitoring of various health related activities of a person. For example, a WMD may include a medical device that may be used to monitor a person's heart activity. The heart activity monitored by the WMD may be in the form of electrical signals (i.e., electrocardiogram or ECG). The WMD may be in a form factor capable of being worn by a person, whose heart activity is to be monitored. Monitoring of a person's ECG may facilitate detection of heart related issues.

Some WMDs may include the capability of monitoring and treating a person's health condition. An example of a WMD, which may be used to monitor and facilitate treatment of a person's heart activity, may be a cardioverter defibrillator type medical device (e.g., wearable cardioverter defibrillator or WCD). Some examples of WCDs may include a support structure or garment that a person may be able wear. The support structure or garment may include various electronic components to facilitate the functionality of the WCD (e.g., monitoring and providing therapy of the person's heart). For example, the WCD may include ECG electrodes for monitoring the person's heart activity, a couple of therapy electrodes for providing an electrical shock to the person's heart, and various electronic components to facilitate the functionality of the WCD (e.g., power supply, user interface, processor, storage device, etc.). The WCD may be included in the support structure and worn by the person due to a particular heath condition of the person (i.e., the person's heart). However, the person's health condition may change, where the change in the person's health condition may be monitored and/or treated with varying components.

For example, the person, who utilized a WCD, may no longer need the therapy components of the WCD (i.e., the electric shock). However, the person may have an additional or different health condition that may be monitored by a different component of the WMD. Accordingly, health care devices having a variety of monitoring devices may be capable of addressing a variety of health related issues. The variety of monitoring devices may complement each other and may provide a more comprehensive indication of a person's health. These monitoring and/or therapy health devices may be included in a WMD.

All subject matter discussed in this section of this document is not necessarily prior art and may not be presumed to be prior art simply because it is presented in this section. Plus, any reference to any prior art in this description is not and should not be taken as an acknowledgement or any form of suggestion that such prior art forms parts of the common general knowledge in any art in any country. Along these lines, any recognition of problems in the prior art are discussed in this section or associated with such subject matter should not be treated as prior art, unless expressly stated to be prior art. Rather, the discussion of any subject matter in this section should be treated as part of the approach taken towards the particular problem by the inventor(s). This approach in and of itself may also be inventive. Accordingly, the foregoing summary is illustrative only and not intended to be in any way limiting. In addition to the illustrative aspects, embodiments, and features described above, further aspects, embodiments, and features will become apparent by reference to the drawings and the following detailed description.

### SUMMARY

Described herein are various illustrative wearable healthcare systems, which may be worn by a person. Example wearable healthcare systems may include modular systems, which may be segmentable, convertible, and/or reconfigurable based, at least in part, on a health condition of the person for monitoring and/or treatment. Example wearable healthcare systems may include a support structure configured to be worn by the person, a reconfigurable medical device integrated with the support structure. The reconfigurable medical device may be configured to be removeable. Example wearable healthcare system may include a monitoring module communicatively coupled with the reconfigurable medical device, where the monitoring module may be configured to be removeable and may be configured to receive a number of signals corresponding to a health condition of the person. Additionally, example wearable healthcare system may include a therapy module communicatively coupled with the reconfigurable medical device, where the therapy module may be configured to be removeable and may be configured to administer a therapeutic action based, at least in part, on the received plurality of signals.

Additionally, described herein are various wearable modular cardiac therapy and monitoring systems. Example wearable modular cardiac therapy and monitoring systems may include a support structure, where the support structure may be configured to be worn by a patient. Example wearable modular cardiac therapy and monitoring systems may include an electrocardiogram (ECG) electrode module, where the ECG electrode module may be configured to be attachable to the support structure while the support structure is being worn by the patient. Example wearable modular cardiac therapy and monitoring systems may include a defibrillator module coupled to the ECG electrode module, where the defibrillator module may be configured to be removably attached to the support structure while the support structure is being worn by the patient. Additionally, example wearable modular cardiac therapy and monitoring systems may include a monitor module coupled to the ECG electrode module, where the monitor module may be configured to be removably attached to the support structure while the support structure is being worn by the patient during a monitor mode of the system. The monitor module and the defibrillator module may be removably attached to the support structure independently in time.

Some additional examples of wearable healthcare systems may be reconfigurable corresponding to a person's health condition. For example, as a person's health condition changes (e.g., improves or deteriorates), the wearable healthcare system may be reconfigurable corresponding to the person's health condition. That is some features or segments of the wearable healthcare system may be added, converted, substituted, or removed.

The foregoing summary is illustrative only and not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

Subject matter is particularly pointed out and distinctly claimed in the concluding portion of the specification. The foregoing and other features of the present disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

In the drawings:

FIG. 1 illustrates a wearable healthcare system, in accordance with various embodiments;

FIG. 2 illustrates an example of a reconfigurable medical device that may be integrated with a wearable healthcare system, in accordance with various embodiments;

FIG. 3 illustrates a wearable healthcare system having a support structure, in accordance with various embodiments;

FIG. 4 illustrates a wearable healthcare system having a support structure, in accordance with various embodiments;

FIGS. 5A and 5B illustrate some examples of support structures, which may be utilized with various embodiments;

FIG. 6 illustrates integration of a reconfigurable medical device integrated with a mobile structure, in accordance with some embodiments; and

FIG. 7 is a block diagram illustrating components of reconfigurable medical device, in accordance with various embodiments.

### DETAILED DESCRIPTION

The following description sets forth various examples along with specific details to provide a thorough understanding of claimed subject matter. It will be understood by those skilled in the art after review and understanding of the present disclosure, however, that claimed subject matter may be practiced without some or more of the specific details disclosed herein. Further, in some circumstances, well-known methods, procedures, systems, components and/or circuits have not been described in detail in order to avoid unnecessarily obscuring claimed subject matter.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

This disclosure is drawn, inter alia, to apparatus and systems related to a wearable healthcare having various healthcare components that are reconfigurable based, at least in part, on a person's health condition.

In the present disclosure, a wearable healthcare system (from hereon healthcare system) may include one or more reconfigurable medical devices (from hereon medical device), which may be used to facilitate monitoring and treatment of various medical conditions of a person. The healthcare system may include a wearable frame (from hereon, scaffold) upon which various medical devices may be integrated and reconfigured. For example, a healthcare system may include a medical device that may be configured to facilitate monitoring and treatment of potential issues with a person's heart (i.e., the person may have a health condition, where the electrical control system of the heart may malfunction causing the heart to beat irregularly or not at all). Commonly, these types of medical devices may include a defibrillator device (e.g., a wearable cardioverter defibrillator or WCD). In the present disclosure, the WCD may be reconfigurable based, at least in part, on the health condition of the person. Accordingly, the disclosure will be described referencing medical devices, which may be integrated with a medical device (i.e., included in the scaffold of the healthcare system), in accordance with various embodiments.

Briefly, the above mentioned issue with the rate of the heartbeat may be generally referred to as arrhythmia. Arrhythmia may be caused by many factors, but in general, arrhythmia may be caused by a malfunction in the electrical control system of the heart. Some types of arrhythmias may result in inadequate blood flow resulting in reduction or lack of the amount of blood pumped to the various parts of the body. For example, issues with the sinoatrial (SA) node may lead to arrhythmia of some kind. Some arrhythmias may lead to a condition known as sudden cardiac arrest (SCA). In an SCA condition, the heart may fail to pump blood effectively, and as a result, death may occur.

An example type of arrhythmia, which may be associated with SCA, may be a condition known as ventricular fibrillation (VF). VF may be a condition where a ventricle or ventricles, which make up the heart to facilitate the pumping of blood, may make uncoordinated movements instead of steady rhythmic movements. In the VF condition, the heart may not pump adequate amounts of blood or may not pump blood at all, which may eventually lead to death. Another type of arrhythmia, which may be associated with SCA, may be a condition known as ventricular tachycardia (VT).

Turning back to the medical device configured to be utilized to help treat VF by defibrillating the heart, the medical device may be capable of monitoring the electrical signals of the person's heart, and if necessary, administer therapy to the heart in the form of an electric shock. The medical device may monitor the electrical signals and provide the electric shock to the heart externally (i.e., through the surface of a body) via components commonly known as electrodes. The medical device may be in the form of a cardioverter defibrillator. The medical device may be included in the scaffold configured to be worn by the person. In this example, the medical device may help facilitate monitoring the electrical activities of the person's heart and providing the electric shock to the heart in the VF condition. As a result, the medical device may help prevent Sudden Cardiac Death (SCD).

In various embodiments, the medical device may include a variety of components to facilitate functionality of the medical device as a cardioverter defibrillator. For example, the medical device may have a number of electrodes to facilitate monitoring (e.g., monitor electrodes) of the electrical signals of the heart and a couple of electrodes to administer an electric shock as treatment (e.g., therapy electrodes). As part of the monitoring (e.g., arrythmia detection), the medical device may be configured to receive electrocardiogram (ECG) signals from the number of monitor electrodes (e.g., ECG electrodes) on the skin of a person. If the received ECG signals indicate a potential arrythmia event, the medical device may be configured to provide a defibrillating shock to the person's heart via the therapy electrodes. In accordance with various embodiments, as the person's health condition changes (e.g., less risk of an arrythmia event), the medical device may be reconfigured to be more of a monitoring healthcare device/system rather than a monitoring and therapeutic medical device.

The above example of the healthcare system may include a wide variety of medical devices configured for monitoring and/or treatment such as, but not limited to, an impedance monitoring sensor, motion sensor, non-invasive blood pressure (NIBP) sensor, hemodynamic sensor, pulse oximeter (SpO₂) sensor, strain sensor, temperature sensor, moisture/perspiration sensor, and/or any combination thereof. In accordance with various embodiments, these examples of medical devices may be included in a healthcare system configured to be worn by a person (i.e., scaffold). The scaffold may function as a wearable platform to facilitate modular configuration and reconfiguration of the medical devices based, at least in part, on the health condition of the person.

Before turning to the figures, a non-limiting example configurations and utilization of the various embodiments of the present disclosure is described. In the non-limiting example, a wearable healthcare system (healthcare system) may include a support structure (scaffold) configured to be worn by a person. A reconfigurable medical device (medical device) may be integrated with the scaffold. The medical device may be configured to facilitate monitoring and treatment of a person's heart. In one example, the medical device may be a wearable cardioverter defibrillator (WCD) device. The scaffold, having the medical device, may be in wide variety of wearable form factors such as, but not limited to, garments, including vests, shirts, undergarments, t-shirts, wearable frame, etc.

In this example, the medical device may include a number of electrodes to facilitate monitoring of electrical signals (monitor electrodes) from the person's heart and to deliver an electric shock for the defibrillation process (therapy electrodes). Additionally, the medical device may include one or more electronic modules having the electronic components to facilitate the monitoring and/or the treatment of the heart (hereon collectively, medical device monitor). In order to facilitate the monitoring of the activities of the heart and to facilitate the administration of the treatment/therapy of the heart (e.g., an electric shock for defibrillation, cardioversion and/or pacing) via the therapy electrodes, the electrodes may be included in the scaffold proximate to the person's heart and/or close to or on the skin of the person.

The medical device monitor may comprise of various electronic components to facilitate operation of the medical device for both monitoring and therapeutic purposes. For example, the medical device monitor may include a power supply such as, but not limited to, a battery to provide a defibrillator electrical shock to the person via the therapy electrodes. Along with the battery, the medical device monitor may include one or more capacitors as part of a discharge circuit for the shock. Additionally, the medical device monitor may include a user interface such as, but not limited to, a physical button (e.g., response buttons), graphical user interface (e.g., display, interactive and non-interactive), audible interface (e.g., indication sounds), etc. The operation and coordination of the electronic components may be facilitated by a processor included in the medical device monitor being communicatively coupled to the various electronic components to facilitate operation of the medical device. The communicatively coupling may include physical couplings and/or wireless couplings. For example, the medical device may include wireless communication methodologies such as, but not limited to, Wi-Fi, IEEE 802 based, Bluetooth® type, Near Field Communication (NFC), radio-frequency identification (RFID), ad-hoc wireless network solutions (e.g., AirDrop), internet of things (IoT) related communication solutions, mesh local area network (LAN) type (e.g., ZigBee, Bluetooth Low Energy, Z-Wave, 6LoWPAN, Thread, etc.), and any combination thereof. Accordingly, the medical device monitor may be configured to communicate with various remote devices such as, but not limited to, a smart phone type device, a tablet type device, a server type device, cloud networks, and so forth.

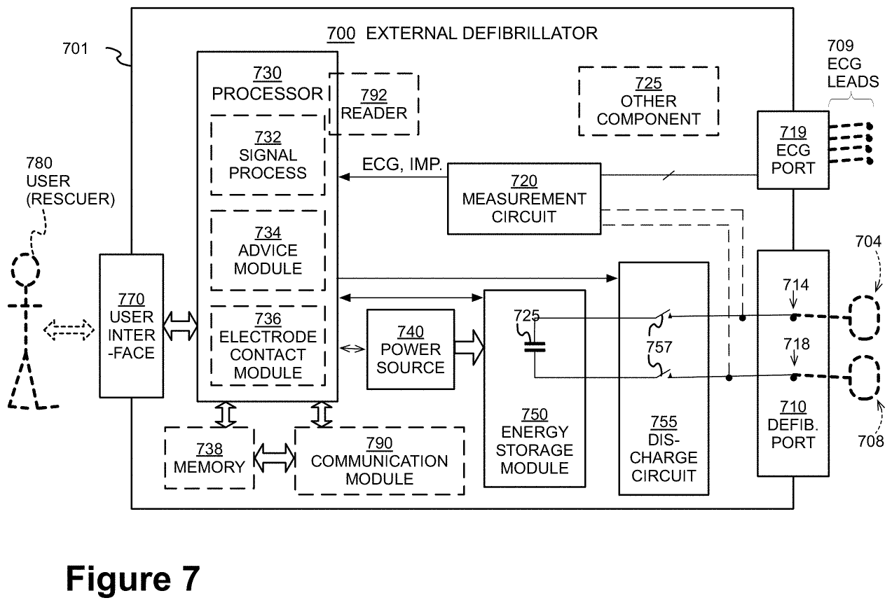

It should be appreciated that after review of this disclosure that the above example components are just a few examples, and accordingly, electronic components of a medical device monitor may include a wide variety of electronic components to facilitate operation of the medical device. Some of details of the medical device will be described below with respect to FIG. 7.

Continuing with the non-limiting scenario, the health condition of the person may have changed over time. That is, the risk of the person experiencing an arrythmia event may have decreased. However, the person's health condition may be such that the person's heart may be monitored for a period of time (i.e., a monitoring period for potential of cardiomyopathy). During the monitoring period, the monitoring components of the medical device may be utilized to monitor the person's heart activity (e.g., monitoring electrodes and its related components), while the therapeutic components may no longer be utilized (e.g., therapy electrodes and its related components). In another example, a person may have received an implanted defibrillator, but healthcare personnel may want to continue to monitor the person's heart activity.

In accordance with various embodiments, the scaffold having the integrated medical device may be reconfigured to remove the therapeutic components (e.g., defibrillator related components), while the monitoring components may be retained to facilitate monitoring of the person's heart (e.g., ECG signal monitoring related components). Since the therapeutic components may have been removed from the scaffold, the scaffold may accommodate components for a variety of other types of medical devices and their corresponding components. For example, a blood oxygen saturation level sensor (e.g., 402) may be disposed in the scaffold. The 402 sensor may be configured to be disposed in a part of the scaffold, which was previously occupied by a component related to therapeutic functionality of the medical device (e.g., therapy electrodes).

In one example, the 402 sensor may be included as part of a self-contained device. The self-contained device may include various electronic components to measure the blood oxygen saturation level (e.g., physiological sensors) and display the measurements on a display of the 402 device. In another example, the 402 sensor may be communicatively coupled to the medical device monitor and transmit the data from the 402 sensor to the medical device monitor (e.g., a processor included in the medical device monitor) to be processed with the measurements being displayed on a display of the medical device monitor. In the self-contained example above, the 402 sensor may include its own power supply, processor, etc. In the communicatively coupled example above, the SpO₂ sensor may utilize the various electronic components of the medical device monitor such as, but not limited to, the power supply, the processor, the communication capabilities (e.g., wireless communication), the storage medium, etc.

In another example, the SpO₂ sensor may be a hybrid of self-contained and communicatively coupled with the medical device monitor. For example, the hybrid SpO₂ sensor may include a processor and a display but may utilize the power supply of the medical device monitor.

Briefly, the blood oxygen saturation level sensor may be implemented in a variety of methodologies such as, but not limited to, measurement of arterial oxygen saturation (SaO₂) levels. Additionally, the SaO₂ levels may be approximated by measurement of peripheral oxygen saturation (SpO₂) levels, which may be measured using a pulse oximeter device. The physiological sensors of the SpO₂ device may be disposed and integrated with the scaffold to facilitate utilization of the measurements of the blood oxygen saturation levels by the reconfigured medical device. Accordingly, the medical device may be considered to be in a monitoring mode.

In the non-limiting scenario, the medical device monitor may be referred to generically as a module having the mechanical and/or electronic components to facilitate the various healthcare related modes described herein. For example, the medical device monitor may include the components to facilitate the utilization of the medical device monitor as a monitoring medical device and/or include components to facilitate communication with blood oxygen saturation level sensor.

Within the scope of this scenario, the health condition of the person may have changed, where the therapeutic action may be potentially necessary (e.g., therapy/treatment mode) again. Accordingly, medical device may be reconfigured to include the therapy related components, where the therapy related components may be integrated back into the scaffold to facilitate the therapeutic action (e.g., the therapy electrodes and their related components).

Additionally, a variety of types of medical devices may be integrated in the scaffold such as, but not limited to, a continuous glucose monitoring (CGM) module. In this example, the CGM may be configured to determine the glucose level of a person, and responsive to the determined glucose level, the CGM may be configured to administer a treatment/therapy (e.g., insulin via an insulin pump). If the person's health condition changes, where the person no longer requires insulin (e.g., glucose level managed by other means), the CGM may be reconfigured to operate without the insulin pump. In some examples, the CGM may be communicatively coupled to the medical device monitor having various components for determining the glucose level of the person. Alternatively, the CGM may be integrated with the scaffold as a separate medical device.

As described, the healthcare system having the scaffold with the integrated medical device may be configured and reconfigured to correspondingly change with changes in the health condition of the person. Additionally, these changes in the health condition may indicate a health condition trend for the person. For example, measurements of the person's health received and analyzed at one point in time may indicate a first status of the person's health. Subsequently, measurements of the person's health received and analyzed at a second point in time may indicate a second status of the person's health. Comparing the first status and the second status may indicate a deterioration of the person's health (e.g., a negative change in the measurements). Alternatively, the first status and the second status may indicate an improvement of the person's health (e.g., a positive change in the measurements). The healthcare system may be reconfigured (i.e., the medical device) to able to correspondingly change with the change in the health status of the person.

As a result, a wearable healthcare system may have a support structure that may be wearable by a person. Integrated with the support structure, a reconfigurable medical device may be utilized to at least independently either monitor various health conditions of the person or monitor and provide therapy for the person. Whether monitoring or monitoring and providing therapy may be based, at least in part, on the health condition of the person, including changes and/or trends in the health condition of the person.

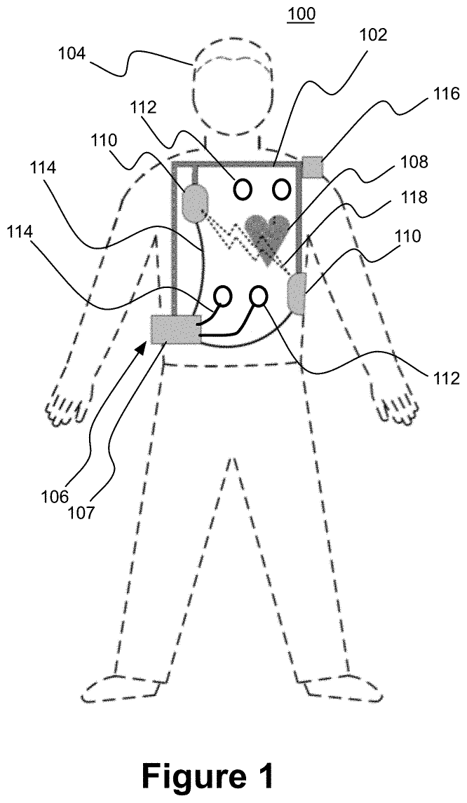

Turning now to FIG. 1, FIG. 1 illustrates a wearable healthcare system, in accordance with various embodiments. In FIG. 1, a wearable healthcare system (healthcare system 100) may include a support structure (scaffold 102). The scaffold 102 may be configured to be worn by a person 104. As shown, a reconfigurable medical device (medical device 106) may be integrated with the scaffold 102. In the example of FIG. 1, the medical device 106 may be configured to facilitate monitoring and treatment of a person's heart such as, but not limited to, a wearable cardioverter defibrillator (WCD). The medical device 106 may include various electronic components to facilitate the functionality of the medical device 106. The various electronic components may include a medical device module (medical device monitor 107). The medical device 106 may include two therapy electrodes (defibrillator electrodes 110) configured to defibrillate a person's heart 108. Additionally, the medical device 106 may include a number of electrodes configured to detect and receive the electrical activity of the person's heart 108 (monitor electrodes 112). The electrical activity of the person's heart 108 may be received as electrocardiogram signals (ECG). As shown, the monitor electrodes 112 and the defibrillator electrodes 110 may be located proximate to the person's heart 108 and chest area. The monitor electrodes 112 and the defibrillator electrodes may be communicatively coupled to the medical device monitor 107 via a number of electrical leads 114.

Additionally, shown in FIG. 1, the scaffold 102 may include a monitoring and/or therapy device separate from the medical device 106. The separate monitoring and/or therapy device may be a wide variety medical device such as, but not limited to, a medical device configured to monitor and treat the health condition related to the glucose level of the person 104 (e.g., continuous glucose monitoring module including an insulin pump (from hereon CGM 116). Accordingly, the healthcare system 100 may include reconfigurable healthcare devices to facilitate physiological measurements of the glucose level of the person 104, which may facilitate determining the health of the person 104, as previously described.

As described in the previous non-limiting scenario, the healthcare system 100 may be configured to be in a monitoring and therapy mode. Accordingly, if the medical device monitor 107 receives an indication that the person's heart 108 may be experiencing an arrhythmia event via the monitoring electrodes 112 (e.g., ECG signals from the heart 108), the medical device monitor 107 may provide a defibrillating shock 118 to the person's heart 108. However, if the health condition of the person 104 has changed to a condition, where the person 104 may no longer need the therapy mode but may need to continue to be monitored, the RMD 106 may be reconfigured for this purpose (e.g., monitoring mode). That is, the various components related to the therapy mode may be removed (e.g., defibrillating electrodes 110 and their respective electrical leads 114). The person may wear the healthcare system 100 with the integrated medical device 106 comfortably without the healthcare system 100 being obtrusive to the person 104. During the monitoring mode, the medical device monitor 107 may receive the ECG signals from the heart 108 via the monitoring electrodes 112. The received ECG signals may be stored, communicatively transmitted, processed, analyzed, and/or any combination thereof. The received ECG signal may provide a trend in the health condition of the person (e.g., improvement in the heart activity), which may result in the person 104 no longer needing to wear the healthcare system 100. Alternatively, the received ECG signal may provide a deterioration of the heart activity, which may result in the medical device 106 being reconfigured to reattach the therapy related components onto the scaffold 102.

Similar to the medical device 106, the CGM 116 may be modular and reconfigurable, in accordance with various embodiments. For example, the CGM 116 may have physiological sensors for measuring the glucose level of the person 104 (i.e., monitoring) and providing insulin (i.e., therapy). The person's health condition may have changed, where the person 104 may no longer need to receive insulin but require glucose monitoring (e.g., monitoring mode). The therapy related components of the CGM 116 (e.g., insulin pump) may be removed (i.e., the CGM 116 may be reconfigured).

As previously described, in one example, the CGM 116 may be communicatively coupled to the medical device monitor 107. In this example, the CGM 116 may have physiological sensors for measuring the glucose level of the person 104 without the need for various other electronic components. Accordingly, the CGM 116 may transmit glucose level measurements to the medical device monitor 107 to be processed and analyzed (e.g., utilize the processors and various electronic components of the medical device monitor 107 including its power supply). The processed and analyzed measurements may be provided to the person 104 via a display (not shown), which may be included in the medical device monitor 107.

In another example, the processed and analyzed measurements may be provided to the person 104 via a display (not shown) included in the CGM 116. In another example, the measured glucose level may be processed and analyzed by the CGM 116. In another example, the measured glucose level may be communicated wirelessly to smart phone to be processed and analyzed. In yet another example, the measured glucose level may be wirelessly communicated to a remote server/computer such as, but not limited to, a server/computer located at a healthcare provider facility.

In yet a further example, the CGM 116 and the medical device monitor 107 may be communicatively coupled to facilitate a hybrid self-contained CGM 116. In this example, the CGM 116 may configured to utilize one or more electronic components included in the medical device monitor 107. As a result, the person. 104 may comfortably wear the healthcare system 100, including the CGM 116, to facilitate measurement a wide variety of health related conditions, in turn, may increase the accuracy of treatment (e.g., defibrillation/insulin) of the person 104.

In some examples, the healthcare system 100 may be configured to be a wearable modular cardiac therapy and monitoring system (from hereon cardiac system). The cardiac system may include a support structure, where the support structure may be configured to be worn by a patient. The cardiac system may include an electrocardiogram (ECG) electrode module, where the ECG electrode module may be configured to be attachable to the support structure, while the support structure is being worn by the patient. In this example, the cardiac system may include a defibrillator module coupled to the ECG electrode module. The defibrillator module may be configured to be removably attached to the support structure, while the support structure is being worn by the patient. As described herein, the cardiac system may include a monitor module coupled to the ECG electrode module. The monitor module may be configured to be removably attached to the support structure, while the support structure is being worn by the patient during a monitor mode of the cardiac system. For this example, the monitor module and the defibrillator module may be removably attached to the support structure independently in time (i.e., monitor mode and/or therapy mode).

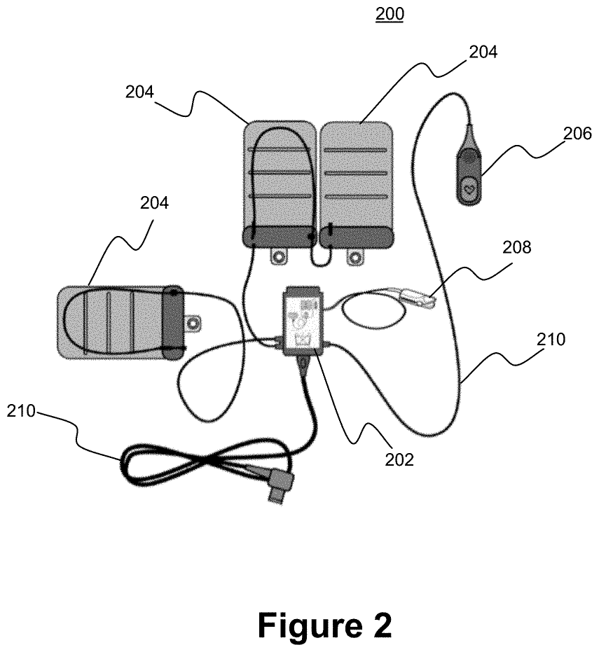

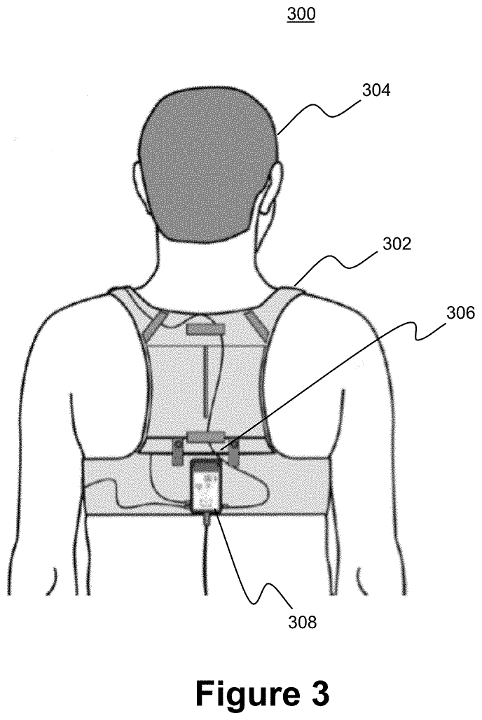

FIG. 2 illustrates an example of a reconfigurable medical device that may be integrated with a wearable healthcare system, in accordance with various embodiments. In FIG. 2, a reconfigurable medical device may be in the form of a WCD 200. The WCD 200 may include a removable junction module (hub 202). Communicatively coupled with the hub 202, the WCD may include three defibrillator electrodes 204, a user interface (e.g., a button 206), and another reconfigurable medical device (e.g., SpO₂ device 208). In FIG. 2, the hub 202 may be utilized to receive cables 210 for interconnecting a defibrillator device with ECG sensors (not shown), the SpO₂ device 208, and the button 206, which may include an abort button to stop an impending shock. Additionally, various optional physiological sensors (e.g., impedance monitoring sensor, motion sensor, non-invasive blood pressure (NIBP) sensor, hemodynamic sensor, strain sensor, temperature sensor, or moisture/perspiration sensor, etc.) may be communicatively coupled to the hub 202. The hub 202 may include various electronic components such as, but not limited to, circuitry for filtering and/or analog-to-digital conversion of ECG and/or other sensor output signals. The modular system 200 may be included in a support structure as shown in FIG. 1 and FIG. 3.

FIG. 3 illustrates a wearable healthcare system having a support structure, in accordance with various embodiments. In FIG. 3, a healthcare system 300 may include a support structure (e.g., garment 302) configured to be worn by a person 304. The garment 302 may include an integrated receptacle 306 configured to receive a hub 308 (e.g., the hub 202 shown in FIG. 2). The hub 308 may be held in the receptacle 306 (i.e., attached) by a variety of methods such as, but not limited to snap fitting, latching, etc.

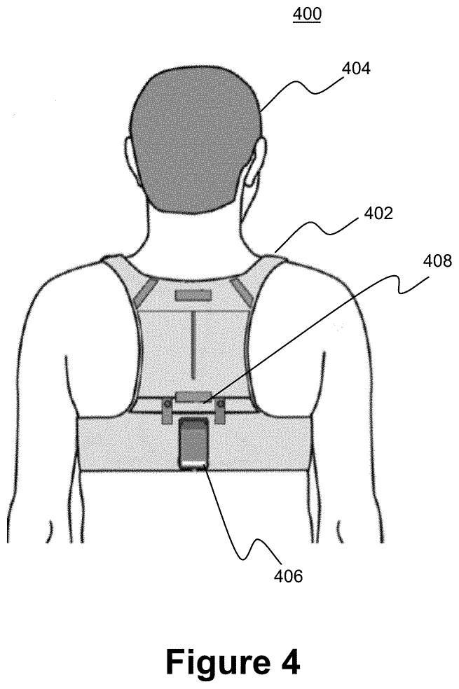

FIG. 4 illustrates a wearable healthcare system having a support structure, in accordance with various embodiments. In FIG. 4, a healthcare system 400 may include a support structure (e.g., garment 402) configured to be worn by a person 404. The healthcare system 400 may include a reconfigurable medical device module 406 in a receptacle 408. The receptacle 408 may be configured to accommodate a hub (such as the hub 202 shown in FIG. 2). However, in FIG. 4, defibrillator functionality may no longer be needed. Accordingly, the hub may be replaced with a monitor module 406 in the receptacle 408.

In one example, the monitor module 406 may include various electronic components (e.g., a processor and other circuitry) to receive signals from ECG electrodes, a pulse oximeter, and other sensors, which may still be attached and/or integrated with the garment 402 and/or the person 404. The monitor module 406 may include a storage medium configured to store the various measurements (e.g., ECG and/or blood oxygen saturation level, etc.). That is, the monitor module 406 may be configured to process the various measurements and store the health parameters of the person 404 such as, but not limited to, ECG signals, heart rate, SpO₂, respiration rate, step count, etc.

In some examples, the monitor module 406 may be a smart device type such as, but not limited to, a smart phone type, a tablet type, and/or a smart watch type having health tracking capabilities (e.g., generate health status, trends, etc. and/or statistics related to the person's activity, sleep, wear time, etc.). Accordingly, some of the data may be processed and communicated to a remote server and/or to cloud storage. Alternatively, raw health related data may be communicated to a remote server to be processed and analyzed. This communication may be in a variety of forms such as wired and/or wireless.

The scope of the disclosure may include that the hub 202 (shown in FIG. 2) may also include various communication capabilities. Accordingly, the hub 202 may include the capabilities of communicating data to a remote server and/or to cloud storage.

The communication capabilities may facilitate remote healthcare services such as, but not limited to, telemedicine. For example, a person's medical personnel may have determined that the person has a high-risk period for VF during which time, the person should be utilizing a WCD. Additionally, there may be a low risk period during which time, the person should be monitored. The WCD system along with a monitor module may be then be provided to the person. The person may utilize the system in the defibrillator configuration of FIG. 3 during the high-risk period. Subsequently, during the low-risk period, the person may reconfigure the system into the monitor configuration of FIG. 4. Once the monitoring period is completed the person may provide the WCD system and the monitor module to the medical personnel or to the vendor of the WCD system and monitor module or to some other designated entity. Alternatively, the information (data) in the WCD system and the monitor module may be communicated (i.e., transmitted) to the medical personnel either via wired and/or wireless communication medium.

In some examples, the WCD system and the monitor module may include storage mediums to store various health related data. The storage medium may be removable and/or non-removeable. The storage medium may be accessed to retrieve the data.

In some examples, the WCD system may be configured to issue prompts to bystanders to place a person's fingertip into/onto the pulse oximeter sensor when the person is determined to be unconscious. For example, some WCD systems include prompting for bystanders to perform CPR on a person, and the pulse oximeter may be utilized to monitor the effectiveness of the CPR and provide CPR feedback to the bystander performing the CPR.

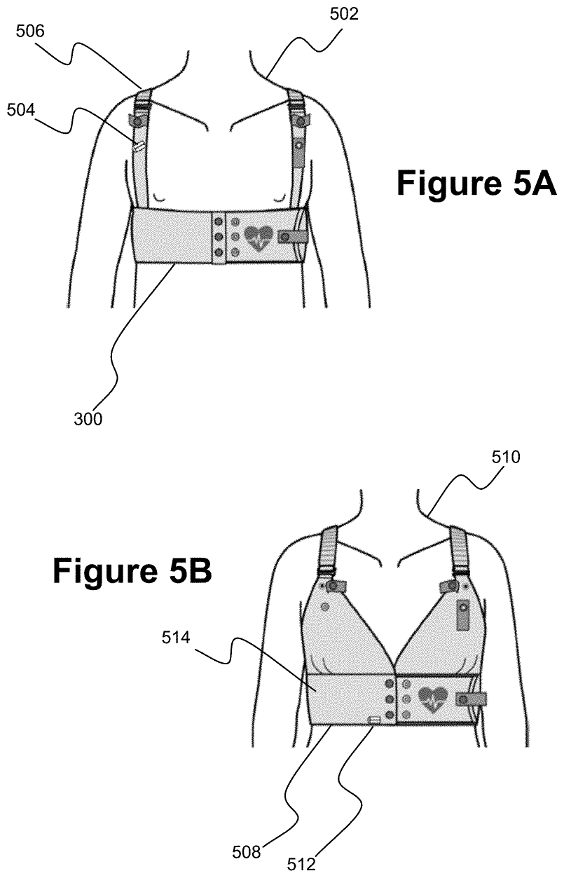

FIGS. 5A and 5B illustrate some examples of support structures, which may be utilized with various embodiments. In FIG. 5A, a first example of a support structure 500 may be configured to be worn by a person 502. The support structure 500 may include a WCD, where the various components of a WCD including a WCD monitor (see FIG. 1) may be integrated into the support structure 500 (i.e., discreetly hidden from view). Additionally, shown in FIG. 5A, the support structure 500 may include a reconfigurable pulse oximeter (SpO₂ device 504). The SpO₂ device 504 may be integrated into the support structure 500, in accordance with various embodiments. As shown, the SpO₂ device 504 may be located and positioned on the support structure 500 to facilitate ease of utilization (i.e., placing a finger into the SpO₂ device 504 with minimal adjustment of the person's clothing). In this example, the SpO₂ device 504 may be located on an over the shoulder strap 506. Having the SpO₂ device 504 located on the shoulder strap 506 may position the SpO₂ device 504 proximate to the person's neck area, where the opening of the SpO₂ device 504 may be directed towards the person's neck. As a result, the person 502 may easily place their left hand finger into a space configured to accommodate of the SpO₂ device 504 discretely underneath or close to a collar area. For example, if the person 502 was wearing some clothing with a collar, the person 502 may discretely place their fingertip into the SpO₂ device 504.

In FIG. 5B, a second example of a support structure 508 may be configured to be worn by a person 510. As with FIG. 5A, the support structure 508 may include a WCD, where the various components of a WCD including a WCD monitor (see FIG. 1) may be integrated into the support structure 508 (i.e., discreetly hidden from view). Additionally, shown in FIG. 5B, the support structure 508 may include a SpO₂ device 512. As with FIG. 5A, in FIG. 5B, the SpO₂ device 512 may be located and positioned on the support structure 508 to facilitate ease of utilization (i.e., placing a finger into the pulse oximeter device with minimal adjustment of the person's clothing). In this example, the SpO₂ device 512 may be located on a portion of the support structure configured to encompass a torso portion of the person 510 (e.g., belt portion 514). Having the SpO₂ device 512 located on the belt portion 514 may position the SpO₂ device 512 in a location, where the person 510 may be able to place their right hand finger into a space configured to accommodate of the SpO₂ device 512 discretely underneath their clothing. For example, the person 510 may be able to lift the bottom portion of their article of clothing and discretely place their fingertip into the SpO₂ device 512.

In other embodiments, a variety of support structures may be utilized to include reconfigurable medical devices. For example, a support structure may be in the form of a garment that may be worn under a person's clothing. In this example, a medical device may be located proximate to the end of a sleeve facilitating easy access to the space configured to accommodate (e.g., a finger in an SpO₂ device sensors).

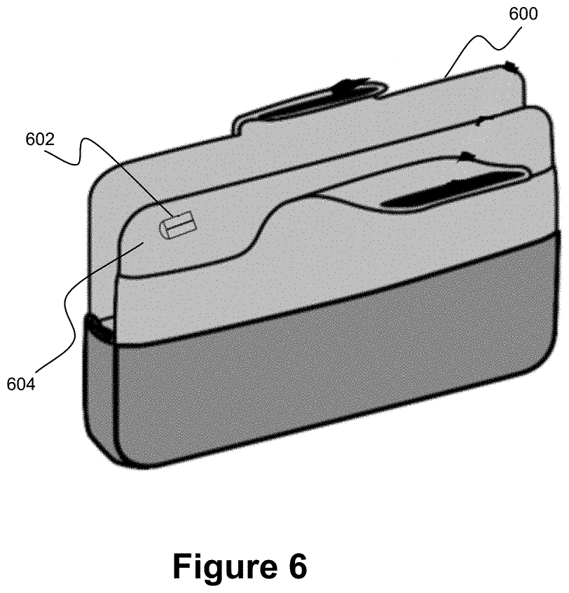

FIG. 6 illustrates integration of a reconfigurable medical device integrated with a mobile structure, in accordance with some embodiments. In FIG. 6, a carry pack 600 may include a medical device 602 (e.g., SpO₂ device). Additionally, the carry pack 600 may be configured to hold a medical device monitor as shown in FIG. 1, where the carry pack 600 may be worn by a person by being attachable (e.g., clipped) to a belt worn by the person or carried using shoulder straps. The medical device 602 may be disposed on an outer surface 604 of the carry pack 600. The medical device 602 may be communicatively coupled to the medical device monitor within the carry pack 600. The person may simply insert utilize the medical device 602 on the outer surface 604 (e.g., a physiological sensor) of the carry pack 600 and take a health condition related measurement. The measurement may be transmitted to the medical device monitor to be processed and analyzed.

FIG. 7 is a block diagram illustrating components of reconfigurable medical device, in accordance with various embodiments. These components may be, for example, components of medical device such as, but not limited to, a WCD 106, 200, 300 (shown in FIGS. 1, 2, and 3).

The defibrillator device 700 may be some of the above examples of a one or more modules for the medical device (e.g., medical device monitor 107 shown in FIG. 1) intended for use by a user 780 (e.g., a wearer, person, patient, etc.). The defibrillator device 700 may typically include a defibrillation port 710, such as a socket in housing 701. The defibrillation port 710 may include nodes 714 and 718. One or more electrodes 704 and 708, which may be removably plugged into the defibrillation port 710, so as to make electrical contact with nodes 714 and 718, respectively. It may also be possible that the electrodes 704 and 708 may be connected continuously to the defibrillation port 710, etc. Either way, the defibrillation port 710 may be used for guiding via the electrodes 704 and 708 to a person 704 an electrical charge that may have been stored in the defibrillator device 700, as described herein.

The defibrillator device 700 may also have an ECG port 719 in the housing 701, for receiving ECG cables 709. The ECG cables 709 may facilitate sensing of an ECG signal (e.g., a 12-lead signal or from a different number of lead signals). Moreover, a defibrillator-monitor could have additional ports (not shown) making the defibrillator device 700 more reconfigurable, and the other component 725 may be configured to filter the ECG signal (e.g., application of at least one filter to the signal to help facilitate removal of artifacts such as, but not limited to, chest compression due to chest compressions being delivered to the person).

The defibrillator 700 also may include a measurement circuit 720. The measurement circuit 720 may receive physiological signals from the ECG port 719, and also from other ports, if provided. The circuit 720 may render detected physiological signals and their corresponding information. The information may be in the form of data, or other signals, etc.

If the defibrillator 700 is configured as a reconfigurable WCD type device as described herein, ECG port 719 may not be present. The measurement circuit 720 may obtain physiological signals through the nodes 714 and 718 instead, when the electrodes 704 and 1108 are attached to the person 704. In these cases, a person's ECG signal may be detected as a voltage difference between the electrodes 704 and 708. Additionally, the impedance between the electrodes 704 and 708 may be detected, among other things, whether the electrodes 704 and 708 have been inadvertently disconnected from the person.

The defibrillator 700 may also include a processor 730. The processor 730 may be implemented in a wide variety of manners for causing actions and operations to be performed. Some examples may include digital and/or analog processors such as microprocessors and digital-signal processors (DSPs), controllers such as microcontrollers, software running in a machine environment, programmable circuits such as Field Programmable Gate Arrays (FPGAs), Field-Programmable Analog Arrays (FPAAs), Programmable Logic Devices (PLDs), Application Specific Integrated Circuits (ASICs), and so on or any combination thereof.

The processor 730 may include a number of modules. One example module may be a detection module 732, which may detect outputs from the measurement circuit 720. The detection module 732 may include a VF detector. Accordingly, the person's detected ECG may be utilized to help determine whether the person is experiencing ventricular fibrillation (VF).

In another example module may be an advice module 734, which may provide advice based, at least in part, on outputs of detection module 732. The advice module 734 may include an algorithm such as, but not limited to, Shock Advisory Algorithm, implement decision rules, and so on. For example, the advice may be to shock, to not shock, to administer other forms of therapy, and so on. If the advice is to shock, some defibrillator examples may report the advice to the user, and prompt them to do it. In other examples, the defibrillator device may execute the advice by administering the shock. If the advice is to administer CPR, the defibrillator 700 may further issue prompts for administrating CPR, and so forth.

The processor 730 may include additional modules, such as module 736 for various other functions. Additionally, if other component 725 is provided, it may be operated in part by processor 730, etc.

In an example, the defibrillator device 700 may include a memory 738, which may work together with the processor 730. The memory 738 may be implemented in a wide variety of manners. For example, the memory 738 may be implemented such as, but not limited to, nonvolatile memories (NVM), read-only memories (ROM), random access memories (RAM), and so forth or any combination thereof. The memory 738 may can include programs for the processor 730, and so on. The programs may include operational programs execution by the processor 730 and may also include protocols and methodologies that decisions may be made by advice module 734. Additionally, the memory 738 may store various prompts for the user 780, etc. Moreover, the memory 738 may store a wide variety of information (i.e., data) such as, but not limited to information regarding the person.

The defibrillator 700 may also include a power source 740. In order to facilitate portability of defibrillator device 700, the power source 740 may include a battery type device. A battery type device may be implemented as a battery pack, which may be rechargeable or not be rechargeable. At times, a combination of rechargeable and non-rechargeable battery packs may be utilized. Additionally, the power source may be configured to be modified to accommodate the power level demands (e.g., monitoring mode without therapy or vice versa). Examples of power source 740 may include AC power override, where AC power may be available, and so on. In some examples, the processor 730 may control the power source 740.

Additionally, the defibrillator device 700 may include a configurable energy storage module 750. The configurable energy storage module 750 may be configured to store some electrical energy (e.g., when preparing for sudden discharge to administer a shock). The configurable energy storage module 750 may be charged from the power source 740 to an appropriate level of energy, as may be controlled by the processor 730. In some implementations, the configurable energy storage module 750 may include one or more capacitors 752, and the like.

The defibrillator 700 may include a discharge circuit 755. The discharge circuit 755 may be controlled to facilitate discharging of the energy stored in energy storage module 750 to the nodes 714 and 718, and also to electrodes 704 and 708. The discharge circuit 755 may include one or more switches 757. The one or more switches 757 may be configured in a number of manners such as, but not limited to, an H-bridge, and so forth.

The defibrillator device 700 may further include a user interface 770 for the user 780. The user interface 770 may be implemented in a variety of manners. For example, the user interface 770 may include a display screen capable of displaying what is detected and measured, provide visual feedback to the user 780 for their resuscitation attempts, and so forth. The user interface 770 may also include an audio output such as, but not limited to, a speaker to issue audio prompts, etc. The user interface 770 may additionally include various control devices such as, but not limited to, pushbuttons, touch display, and so forth. Additionally, the discharge circuit 755 may be controlled by the processor 730 or directly by the user 780 via the user interface 770, and so forth.

Additionally, the defibrillator device 700 may include other components. For example, a communication module 790 may be provided for communicating with other machines and/or the electrodes. Such communication may be performed wirelessly, or via wire, or by infrared communication, and so forth. Accordingly, information may be communicated, such as person data, incident information, therapy attempted, CPR performance, ECG information, and so forth.

The above described components may be configured and reconfigured, in accordance with various embodiments. For example, monitoring mode or monitoring and therapy mode.

It should be appreciated after review of this disclosure that it is contemplated within the scope and spirit of the present disclosure that the claimed subject matter may include a wide variety of healthcare devices. Accordingly, the claimed subject matter is not limited in these respects.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Reference in the specification to “an implementation,” “one implementation,” “some implementations,” or “other implementations” may mean that a particular feature, structure, or characteristic described in connection with one or more implementations may be included in at least some implementations, but not necessarily in all implementations. The various appearances of “an implementation,” “one implementation,” or “some implementations” in the preceding description are not necessarily all referring to the same implementations.

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter is not limited to the particular examples disclosed, but that such claimed subject matter also may include all implementations falling within the scope of the appended claims, and equivalents thereof.

What is claimed:

1. A wearable healthcare system comprising:
a support structure, the support structure configured to be worn by a person;
a reconfigurable medical device integrated with the support structure, the reconfigurable medical device configured to be removeable;
a monitoring module communicatively coupled with the reconfigurable medical device, the monitoring module configured to be removeable and configured to receive a plurality of signals corresponding to a health condition of the person; and
a therapy module communicatively coupled with the reconfigurable medical device, the therapy module configured to be removeable and configured to administer a therapeutic action based, at least in part, on the received plurality of signals.

2. The wearable healthcare system of claim 1, wherein the support structure comprises a scaffold frame.

3. The wearable healthcare system of claim 2, wherein the scaffold frame comprises an attachment mechanism configured to support at least one of the reconfigurable medical device, the monitoring module, and/or the therapy module.

4. The wearable healthcare system of claim 1, wherein the support structure comprises a garment.

5. The wearable healthcare system of claim 1, wherein the reconfigurable medical device comprises a medical modular junction hub.

6. The wearable healthcare system of claim 1, wherein the monitoring module comprises a plurality of electrocardiogram (ECG) electrodes.

7. The wearable healthcare system of claim 1, wherein the monitoring module comprises a physiological sensor.

8. The wearable healthcare system of claim 7, wherein the physiological sensor comprises at least one of impedance monitoring sensor, motion sensor, non-invasive blood pressure (NIBP) sensor, hemodynamic sensor, pulse oximeter (SpO₂) sensor, strain sensor, temperature sensor, or moisture/perspiration sensor.

9. The wearable healthcare system of claim 8, wherein the physiological sensor comprises a continuous glucose monitoring (CGM) module.

10. The wearable healthcare system of claim 9, wherein the therapy module comprises an insulin pump.

11. The wearable healthcare system of claim 1, wherein the therapy module comprises a defibrillator module, the defibrillator module configured to provide a defibrillating electrical shock to the person.

12. The wearable healthcare system of claim 1, wherein the monitor module and the therapy module comprise healthcare modules configured to operate in an independent manner.

13. The wearable healthcare system of claim 1, wherein the monitor module and the therapy module comprise healthcare modules configured to operate in a dependent manner.

14. The wearable healthcare system of claim 1, wherein the reconfigurable medical device comprises the reconfigurable medical device configured to communicate wirelessly with a server.

15. A wearable modular cardiac therapy and monitoring system, the system comprising:
a support structure, the support structure configured to be worn by a patient;
an electrocardiogram (ECG) electrode module, the ECG electrode module configured to be attachable to the support structure while the support structure is being worn by the patient;
a defibrillator module coupled to the ECG electrode module, the defibrillator module configured to be removably attached to the support structure while the support structure is being worn by the patient; and
a monitor module coupled to the ECG electrode module, the monitor module configured to be removably attached to the support structure while the support structure is being worn by the patient during a monitor mode of the system, wherein the monitor module and the defibrillator module are removably attached to the support structure independently in time.

16. The therapy and monitoring system of claim 15 further comprising a pacing module, the pacing module configured to be removably attached to the support structure during a pacing mode of the system.

17. The therapy and monitoring system of claim 15 further comprising a non-ECG sensor module configured to be attached to the monitor module.

18. The therapy and monitoring system of claim 17, wherein the non-ECG sensor module is removably attached to the support structure using a therapy pad receptacle of the support structure.
